(19) United States
(12) Patent Application Publication
(10) Pub. No.: US 2026/0263785 A1
(43) Pub. Date: Sep. 10, 2026

(54) EXPANDING TEMPORARY PACING LEAD

(71) Applicant: Nextern Innovation, LLC, Maple Grove, MN (US)

(72) Inventors: Wesley Robert Pedersen, Minneapolis, MN (US); William J. Drasler, Minnetonka, MN (US); Paul Sorajja, Minneapolis, MN (US)

(73) Assignee: Nextern Innovation, LLC, Maple Grove, MN (US)

(21) Appl. No.: 19/121,958

(22) PCT Filed: Nov. 9, 2023
(86) PCT No.: PCT/US2023/079223

§ 371 Date: Apr. 17, 2025

Related U.S. Application Data
(60) Provisional application No. 63/383,037, filed on Nov. 9, 2022.

(51) Int. Cl. A61N 1/05; A61N 1/362; A61N 1/372

(52) U.S. Cl. CPC A61N 1/0573; A61N 1/362; A61N 1/372

(57) ABSTRACT

Apparatus and associated methods relate to a temporary pacing device. In an illustrative example, a temporary pacing device may include a device body that includes an upper body and a lower body at a distal region. For example, the distal region may include an electrode configured to contact a wall tissue and conduct a voltage at the wall tissue (e.g., for pacing a heart). The device body, for example, may include a coupling system. The coupling system may include a loop control member that may extend longitudinally within the device body. For example, the loop control member may be disposed between the upper body and the lower body. When the loop control member is activated, the coupling system induces the upper body and the lower body to separate to form a loop. Various embodiments may advantageously maintain a firm contact between the electrode and the wall tissue.

### CROSS-REFERENCE TO RELATED APPLICATIONS

This application claims the benefit of U.S. Provisional Application Ser. No. 63/383,037, titled “EXPANDING TEMPORARY PACING LEAD,” filed by Wesley Robert Pedersen, et al., on Nov. 9, 2022.

This application incorporates the entire contents of the foregoing application(s) herein by reference.

The subject matter of this application may have common inventorship with and/or may be related to the subject matter of the following:
U.S. application Ser. No. 17/004,520, titled “Temporary Pacing Lead,” filed by Wesley Robert Pedersen, et al., on Aug. 27, 2020, and issued as U.S. Pat. No. 11,642,518 on May 9, 2023;
U.S. application Ser. No. 15/642,084, titled “Temporary Pacing Lead,” filed by Wesley Robert Pedersen, et al., on Jul. 5, 2017, and issued as U.S. Pat. No. 10,773,076 on Sept. 15, 2020;
U.S. application Ser. No. 17/075,409, titled “Curled Shaft Temporary Pacing Lead,” filed by Wesley Robert Pederson, et al., on Oct. 20, 2020;
U.S. application Ser. No. 13/108,938, titled “Valvuloplasty Catheter and Methods,” filed by William Drasler, et al., on May 16, 2011, and issued as U.S. Pat. No. 8,900,264 issued on Dec. 2, 2014;
U.S. application Ser. No. 16/008,562, titled “Cardiac Stimulation System,” filed by William Drasler, et al., on Jun. 14, 2018; and,
U.S. application Ser. No. 17/597,883, titled “Devices and Methods for Guide Wire Placement, filed by Paul Sorajja, et al., on Jul. 31, 2020.

This application incorporates the entire contents of the foregoing application(s) herein by reference.

### TECHNICAL FIELD

Various embodiments relate generally to implantable pacing leads for stimulating body tissues and/or sensing physiological attributes, for example, including a lead or catheter having electrical stimulation capabilities suitable for delivering electrical stimulations to a wall of a body chamber, hollow organ, surface of a tissue member.

### BACKGROUND

Pace leads may be essential components of cardiac rhythm management systems. For example, some pace lead systems may be designed to monitor and regulate the electrical impulses within a patient's heart.

In cardiac pacing, a pace lead may, for example, include a specialized electrical wire designed to deliver controlled electrical impulses to the heart muscle. For example, predetermined impulses may be delivered at a specific rate, ensuring that the heart maintains an optimal rhythm and heart rate.

In some examples, development of pace lead systems and associated technologies may improve management of various cardiac conditions. Some devices may be indispensable tools in the field of cardiology, offering life-saving interventions for patients with arrhythmias, heart block, or other rhythm disorders.

### SUMMARY

Apparatus and associated methods relate to a temporary pacing device. In an illustrative example, a temporary pacing device may include a device body that includes a first body and a second body at a distal region. For example, the distal region may include an electrode configured to contact a wall tissue and conduct a voltage at the wall tissue (e.g., for pacing a heart). The device body, for example, may include a coupling system. The coupling system may include a loop control member that may extend longitudinally within the device body. For example, the loop control member may be disposed between the first body and the second body. When the loop control member is activated, the coupling system operates the first body and the second body to separate to form a loop. Various embodiments may advantageously maintain a firm contact between the electrode and the wall tissue.

Various embodiments may achieve one or more advantages. For example, some embodiments may advantageously assist in return of the distal region to a linear shape upon removal of tension to the loop fibers or wires. Some embodiments, for example, may advantageously provide the distal region with a planar shape when it is formed in a circular shape upon application of tension to the loop fiber or wires. Some embodiments may, for example, advantageously reduce or eliminate accidental snagging. For example, some embodiments may advantageously safely contact the apical region of the ventricle without concern for potential perforation of the myocardial wall near the apex. Some embodiments may, for example, advantageously increase its surface area for contact with the myocardial tissues. For example, some embodiments may advantageously allow an atraumatic way of advancing the distal region within a body cavity. Some embodiments may, for example, advantageously prevent perforation of a body tissue (e.g., the apex of the cardiac ventricle). For example, some embodiments may advantageously prevent migration of the distal region.

The details of various embodiments are set forth in the accompanying drawings and the description below. Other features and advantages will be apparent from the description and drawings, and from the claims.

### BRIEF DESCRIPTION OF THE DRAWINGS

FIG. 1A depicts an exemplary temporary pacing lead in a linear mode.

FIG. 1B depicts an exemplary temporary pacing lead in an expanded mode, for example, inside the interior of a distal region to form a circular shape.

FIG. 2A and FIG. 2B depict exemplary distal regions of a temporary pacing lead described with reference to FIGS. 1A-B.

FIG. 3A and FIG. 3B depicts embodiments of an exemplary hinge of the temporary pacing lead.

FIG. 4A depicts an exemplary extruded tubing that forms the upper and lower members having a thermally formed narrowed region in a non-bent state.

FIG. 4B depicts an extruded tubing that forms the upper and lower members having a thermally formed narrowed region in a bent state.

FIG. 4C depicts a distal tip that has a smooth (e.g., snag-resistant) contour.

FIG. 5A and FIG. 5B depict exemplary embodiments of the distal region of FIG. 3B and containing loop fibers and electrodes.

FIG. 6A, FIG. 6B, and FIG. 6C show exemplary features of a temporary pacing lead applicable to any of the embodiments shown in FIGS. 1A-5B.

FIG. 7A, FIG. 7B, FIG. 7C, FIG. 7D, FIG. 7E, and FIG. 7F show exemplary fixation electrodes of a temporary pacing lead.

FIG. 8A and FIG. 8B show the exemplary temporary pacing lead in an expanded configuration located in, for example, the right ventricle of the heart.

FIG. 9A, FIG. 9B, FIG. 9C, FIG. 9D, FIG. 9E, FIG. 9F, and FIG. 9G show exemplary assembling methods of various embodiments of the temporary pacing lead.

FIG. 10 depicts an illustrative method of temporary stimulation.

Like reference symbols in the various drawings indicate like elements.

### DETAILED DESCRIPTION OF ILLUSTRATIVE EMBODIMENTS

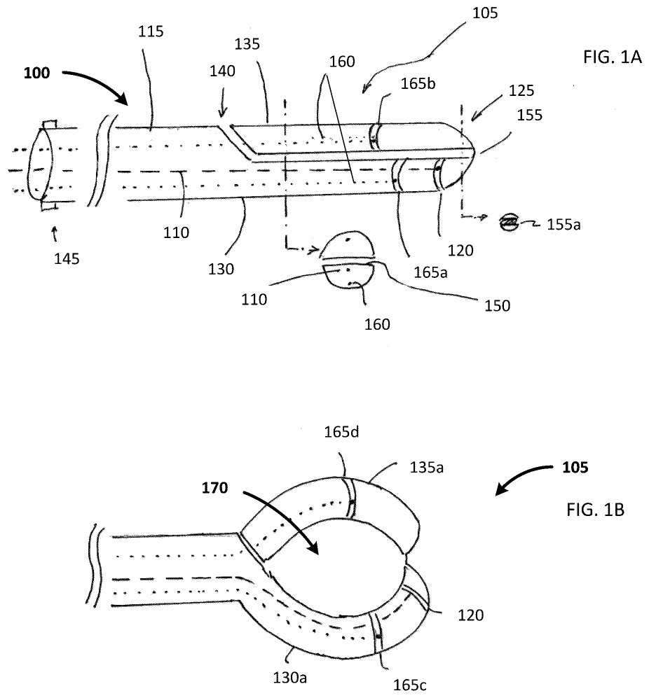

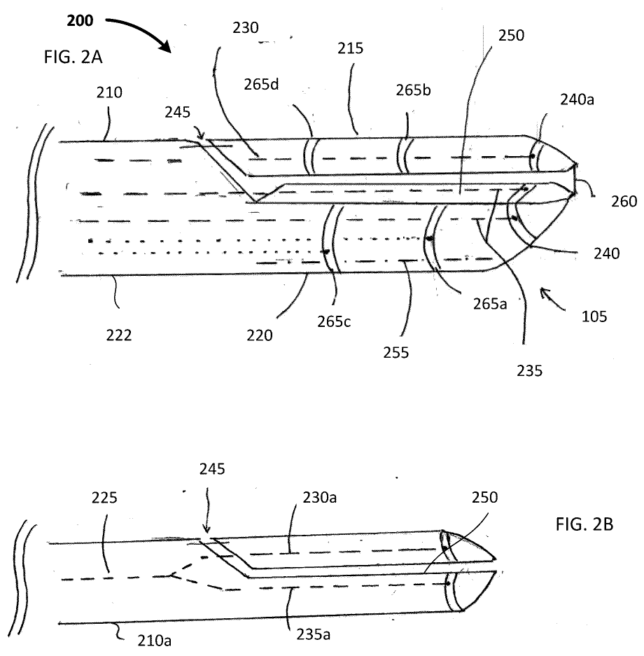

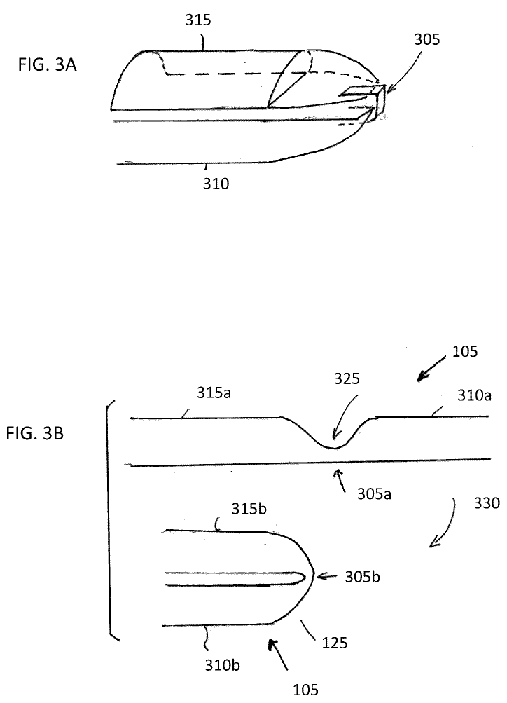

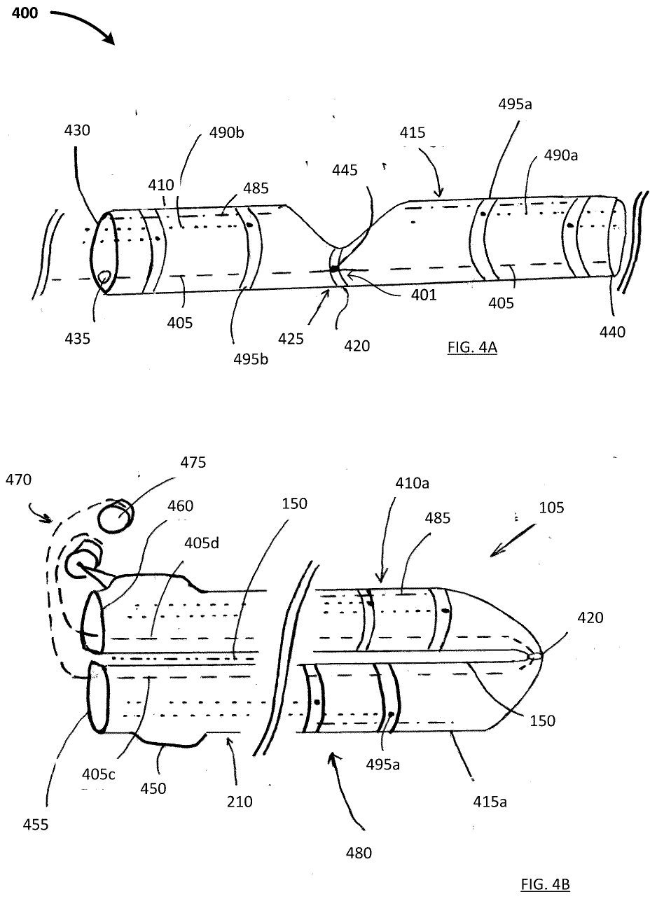

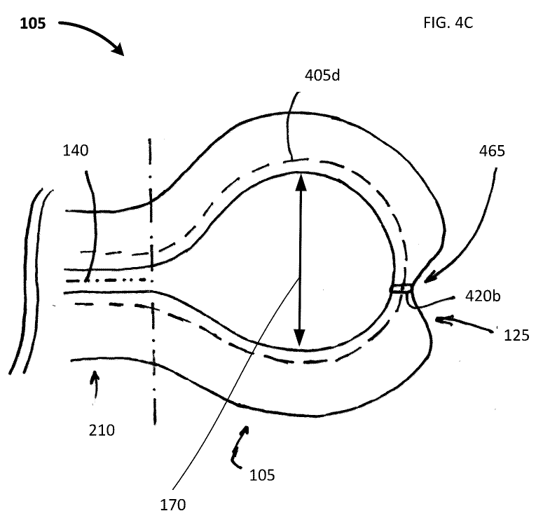

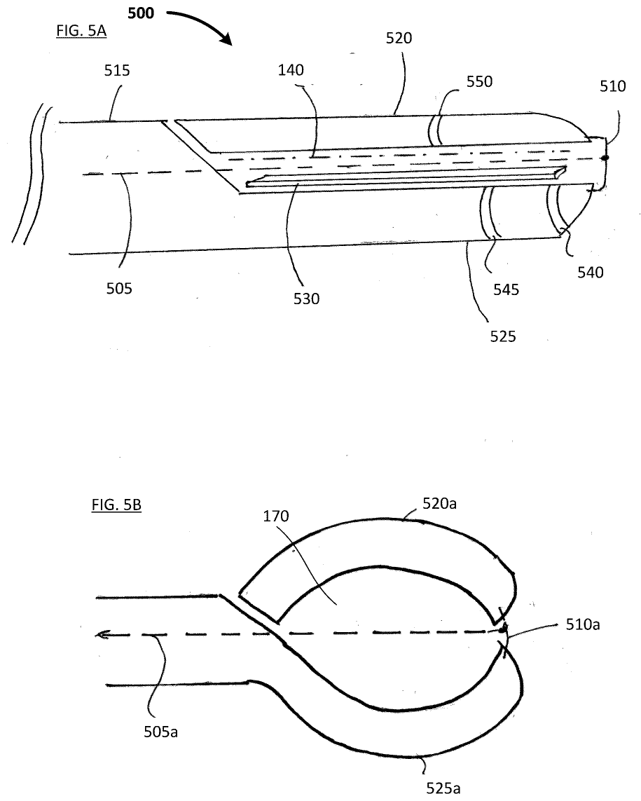

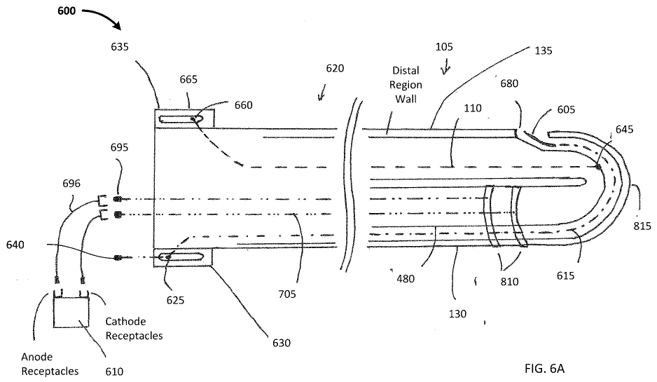

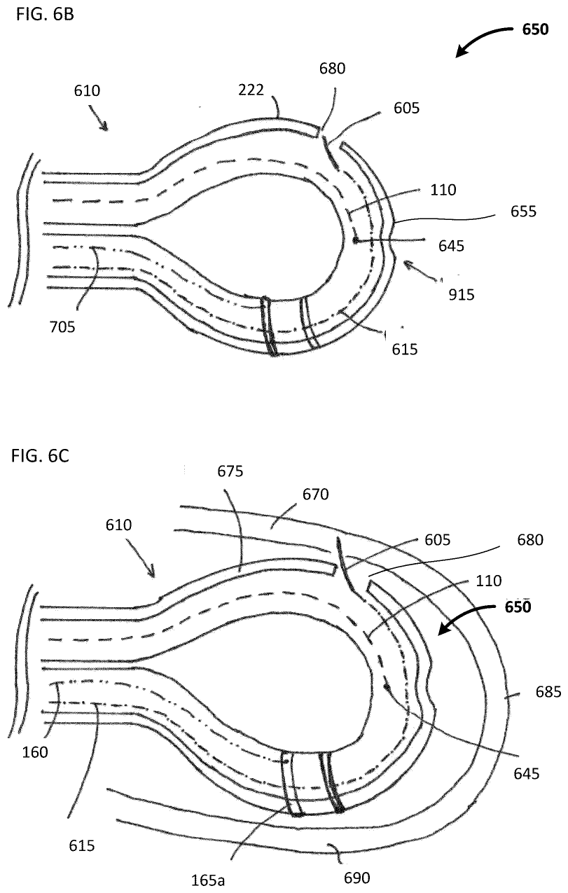

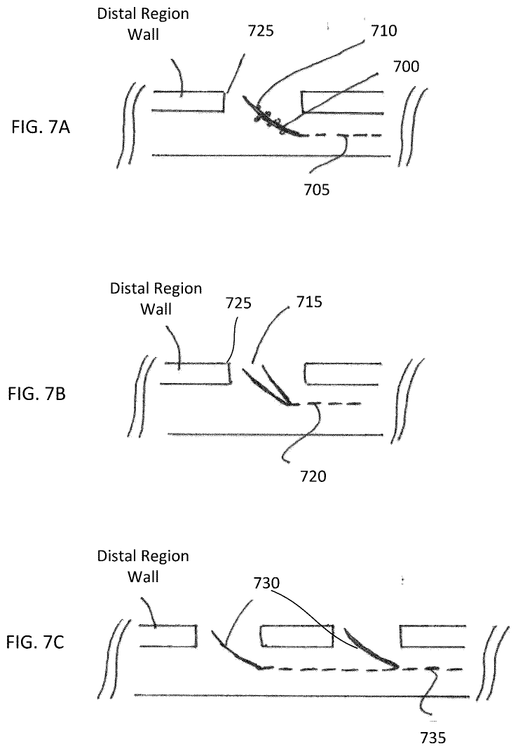

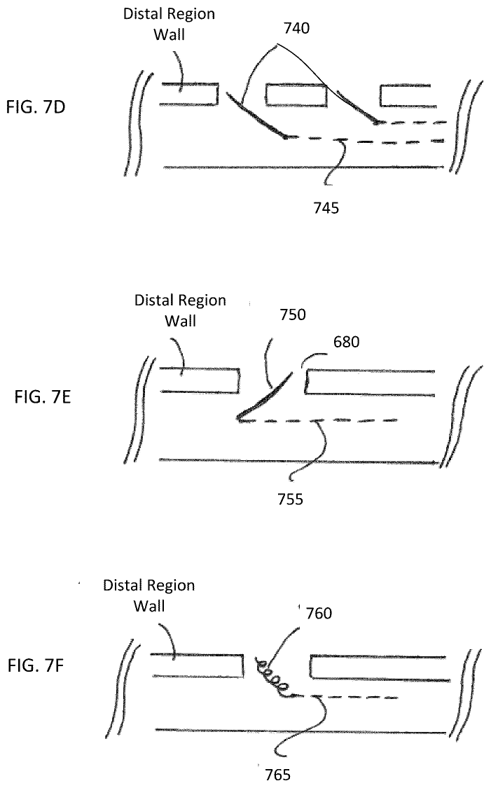

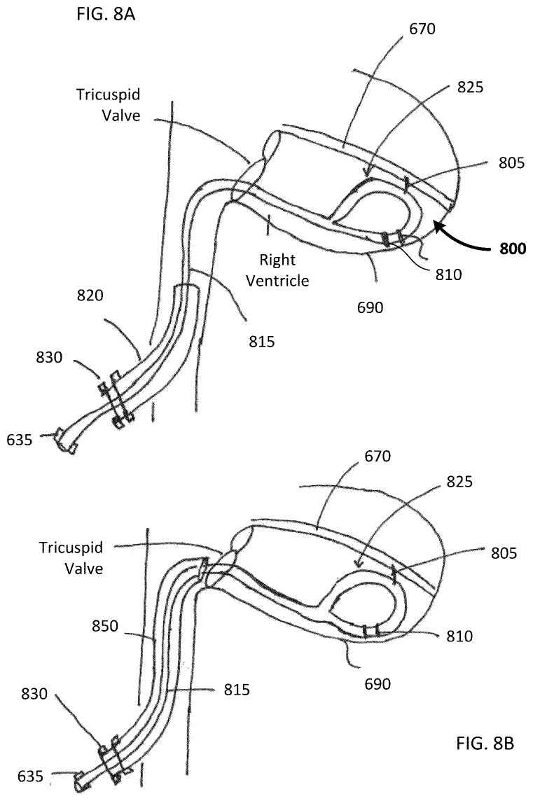

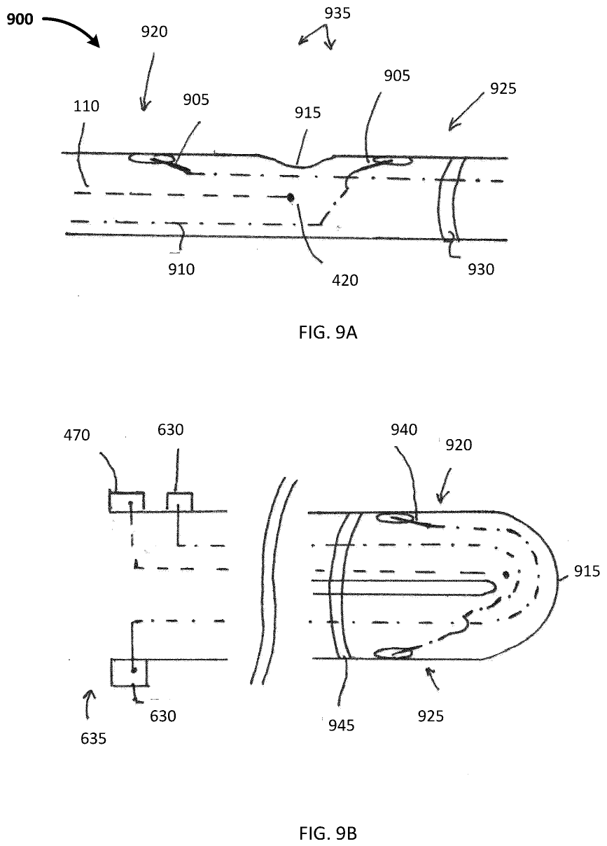

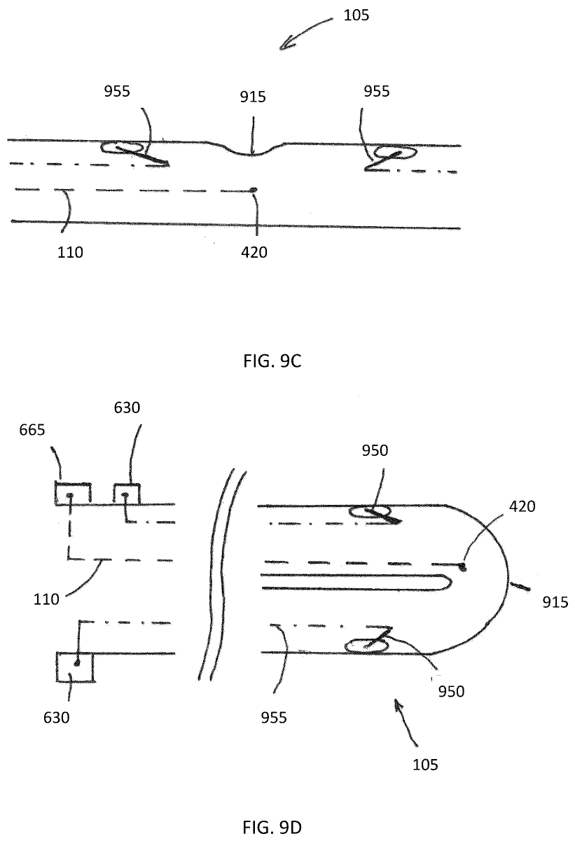

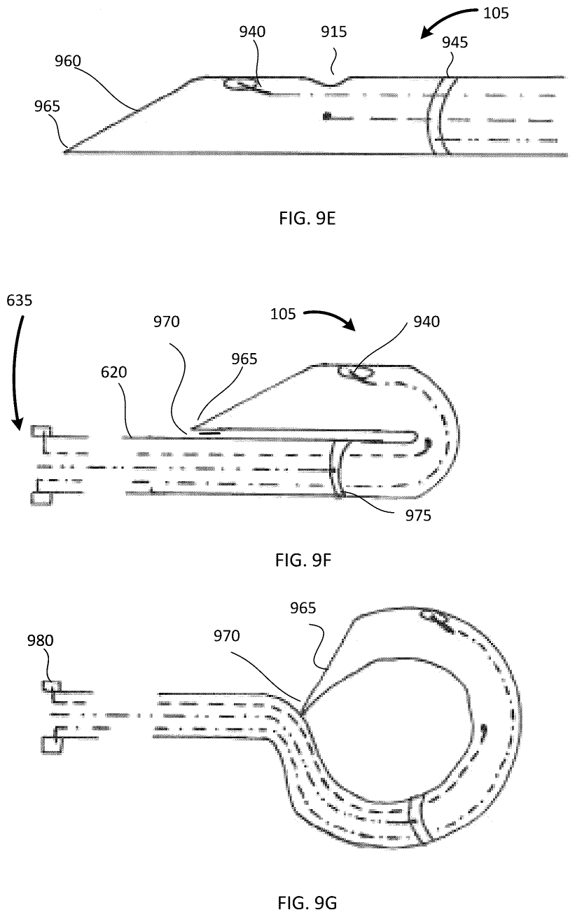

To aid understanding, this document is organized as follows. First, to help introduce discussion of various embodiments, a description of an application of an exemplary expanding temporary pacing lead is introduced with reference to FIGS. 1A-1B. Second, that introduction leads into a description with reference to FIGS. 2A-2B of an exemplary embodiment. Third, various embodiments of a third application are disclosed in FIGS. 3A-4C. Fourth, a fourth application with reference to FIGS. 5A-5B. Fifth, and with reference to FIGS. 6A-C, this document describes exemplary features of a temporary pacing lead. Sixth, this disclosure turns to a review and a discussion of various embodiments of exemplary fixation electrodes of a temporary pacing lead with reference to FIGS. 7A-8B. Seventh, the document introduces exemplary assembling methods of various embodiments of the temporary pacing lead with reference to FIGS. 9A-G. Finally, the document discusses further embodiments, exemplary applications and aspects relating to temporary pacing leads. Although various embodiments have been described with reference to the figures, other embodiments are possible.

FIGS. 1A and 1B depicts an exemplary scenario in which a distal region is contoured by an expansion of the temporary pacing lead by an activation mechanism. The expansion may, by way of example and not limitation, contour a linear shape of the temporary pacing lead into a round configuration. The expanded temporary pacing lead may, for example, contact with opposing walls within a right ventricle of a heart.

As shown in FIG. 1A, a temporary pacing lead 100 includes a distal region 105. For example, the distal region 105 may be inserted percutaneously into a patient. The distal region 105 may, for example, be inserted into a vasculature region of the heart, a structure within the heart, and/or any structures within the heart. The insertion of a proximal body of the temporary pacing lead 100 into the vasculature of the body may, by way of example but not limitation, be shown in the cross references, such as, U.S. application Ser. No. 15/642,084, titled “Temporary Pacing Lead,” filed by Wesley Robert Pedersen, et al., on Jul. 5, 2017, and issued as U.S. Pat. No. 10,773,076 on Sept. 15, 2020; and U.S. application Ser. No. 17/075,409, titled “Curled Shaft Temporary Pacing Lead,” filed by Wesley Robert Pederson, et al., on Oct. 20, 2020.

In this example, a loop control member 110 (e.g., a loop fiber) extends within a proximal body 115 of the temporary pacing lead 100 to reach the distal region 105. The loop control member 110 may, by way of example and not limitation, extend in a sliding motion along the proximal body 115 of the pacing lead. For example, the loop control member 110 may attach to an anchor (e.g., ring 120) near or in proximity of a distal tip 125 of the proximal body 115 of the temporary pacing lead 100. The ring 120 may, by way of example and not limitation, include any anchoring member and/or mechanism that may solidly attach near and/or in proximity of the distal tip 125.

In some implementations, the loop control member 110 may be actuated by placing it under tension to form the loop shape shown in FIG. 1B. For example, the loop control member 110 may be placed under compression to cause the distal region 105 to return to a linear shape as shown in FIG. 1A. Various embodiments of an actuation mechanism of the loop control member 110 are described in more detail later in the specification.

In some implementations, the proximal body 115 of the temporary pacing lead 100 may be split to form a lower member 130 and an upper member 135 at the distal region 105. The lower member 130 and upper member 135 may, by way of example and not limitation, be bonded by a bond 140 to the proximal body 115. The bond may, for example, be created by thermal deformation to the proximal body. The lower member 130 and upper member 135 may, by way of example but not by limitation, be D-shaped. The D-shape may, for example, allow the lower member 130 and upper member 135 to have a flat surface 150.

The flat surface 150 of the D shape of the lower member 130 and upper member 135 may be joined and/or placed into contact with each other at the flat surface 150 to provide low profiles as the proximal body 115 is inserted into the distal region 105. In some implementations, the temporary pacing lead 100 may, for example, include at least one loop fiber (e.g., the loop control member 110) in at least one of the upper member 135 or the lower member 130. Some embodiments may, for example, have one or more loop fibers (e.g., the loop control member 110) in the proximal body 115, the upper member 135, and/or the lower member 130.

The loop control member 110 extends through a lumen of the lower member 130 of the proximal body 115 of the temporary pacing lead 100. For example, tension may be applied (e.g., by a manifold 145) to the loop control member 110 which are anchored to the ring 120. In some examples, the tension may be applied to the loop control member 110 that are anchored to one or more other anchoring members. The manifold 145, by way of example and not limitation, may provide a mechanism to which an operator may apply tension to the loop control member 110 that are extending near the flat surface 150 of the lower member 130 and upper member 135. The application of tension to the two loop located near a flat surface 150 of the lower member 130 of the proximal body 115, by way of example and not limitation, may cause the lower member 130 to bend due to the lower member 130 compressing the flat surface 150.

The lower member 130 and upper member 135 of the distal region 105 attaches to a hinge 155 located at the end of the distal tip 125. The hinge 155 may allow the upper member 135 of the proximal body 115 to form a circular bend if the lower member 130 of the proximal body 115 is forms a circular bend. The hinge 155 and the bond 140 may, for example, be configured to constrain the temporary pacing lead 100 in a single plane when expanded. For example, the hinge 155 and the bond 140 may advantageously avoid the temporary pacing lead 100 to form a 3D ‘saddle’shape.

The circular bend created by the loop control member 110, by way of example and not limitation, may bend to shape the distal region 105 to form a circle, ellipse, donut, and/or other curvilinear shape contouring the distal region 105. An exemplary hinge 155a connecting to the lower member 130 and the upper member 135 is shown in FIG. 1A.

The proximal body 115, the lower member 130, and/or the upper member 135 may be wired with electrode connecting wires 160. In various implementations, the electrode connecting wires 160 may be attached to electrodes 165a in the lower member 130 and the upper member 135. In this example, a first electrode 165a is attached to the lower member 130. A second electrode 165b, as shown in this example, is attached to the upper member 135. The first electrode 165a and the second electrode 165b may, for example, be offset to offer a lower profile when the distal region 105 is advancing through the vasculature to reach the heart.

FIG. 1B depicts an exemplary bending of the temporary pacing lead 100 by the lower member 130 and the upper member 135 to form a curvature in the distal region 105. The tension of the loop control member 110 applied to the ring 120 may, by way of example and not limitation, cause the lower member 130 and upper member 135 to compress the flat surface 150 causing a bending of the lower member 130 and upper member 135 to form the expanded configuration of the distal region as shown in FIG. 1B.

In some examples, a bending motion of the temporary pacing lead 100 may create a (e.g., circular) gap 170 (e.g., a separation distance) that is planar between the upper member 135 and the lower member 130. The gap 170, by way of example and not limitation, may be between 2-2.5 ±1 cm. The upper member 135 and the lower member 130 may be bent, for example. For example, the upper member 135 and the lower member 130 may be provided with substantially identical stiffness. The upper member 135 and the lower member 130 may, for example, be smooth. For example, a smooth surface may advantageously be inserted into the heart chamber without damaging the heart tissues. For example, the smooth surface may reduce or prevent accidental snagging of chordae tendineae and/or other structures within the heart and/or vasculature. The upper member 135 and the lower member 130 may, for example, include shaped or curved upper and lower members to advantageously allow enhanced entry into branched blood vessels of the body.

As shown in FIG. 1B, an expanded lower member 130a and an expanded upper member 135a may also expand the second electrode 165b and the first electrode 165a, respectively. The expansion of the first electrode 165a and the second electrode 165b may, by way of example, be shown as an expanded electrode 1 165c and an expanded electrode 2 165d. Some embodiments may, for example, include two or more electrodes attached along each or the upper member 135 and/or the lower member 130.

FIGS. 2A-2B depict an embodiment of a distal region of a temporary pacing lead. The temporary pacing lead may, by way of example and not limitation, be expanded by one or more loop fibers located only in a lower member of a proximal body of the temporary pacing lead. In some implementations, one or more pull wires may be used in both the upper member and the lower member.

FIG. 2A depicts a temporary pacing lead 100 in an unexpanded state. The temporary pacing lead 200 may be inserted with a distal region 105 in a linear configuration as shown. The distal region 105 of the temporary pacing lead 200 may have an upper member 215 and a lower member 220. The lower member 220 may have a lower loop fiber 235 attached to a ring 240 and or other anchor devices located near the distal end of the pacing lead. The upper member 215 may have an upper loop fiber 230 attached to a ring 240a and/or other anchor devices located near the distal end of the pacing lead.

In some embodiments, the lower member 220 and/or upper member 215 may have more than one loop fiber attached to the ring 240 and/or anchor located near the distal end of the temporary pacing lead 200. As shown, the temporary pacing lead 200 includes a pivot 245 connecting the upper member 215 to the proximal body 210.

In this example, the lower member 220 is contiguous with the proximal body 210. For example, the lower member 220 may be reduced in diameter and made into having a D-shape cross-section using a thermal process and/or a skiving step (i.e., cutting away a portion of the cross-section) to reduce its profile. The upper member 215, for example, may be bent back upon itself at the hinge region to form the upper member.

The pivot 245 may, for example, allow for a smooth and continuous bending location between the proximal body 210 and the upper member 215 forming a curvilinear profile (e.g., planar circle) between the upper member and the lower member. For example, the pivot 245 may advantageously allow the upper loop fiber 230 to pass between the upper member 215 and the proximal body 210. The temporary pacing lead 200 also includes a hinge 260. For example, the hinge 260 may be attached to the upper member 215 and the lower member 220 to couple the members together. For example, the hinge 260 may advantageously provide a planar relation between the upper member 215 and the lower member 220.

In some implementations, the pivot 245 and/or the hinge 260 may, by way of example and not limitation, include a separate polymeric ribbon. For example, the pivot 245 and/or the hinge 260 may include a thinned down region between the upper member 215 and the lower member 220. The pivot 245 and/or the hinge 260 may, for example, include a metal ribbon. In some examples, the pivot 245 and/or the hinge 260 may be coupled by a separate composition (e.g., such as Nitinol wire). Some examples may include supporting, reinforcing, and/or spring-like members supporting the pivot 245 and/or the hinge 260. In some embodiments, the pivot 245 and/or the hinge 260 may, for example, be thermally bonded. Some embodiments may, for example, include adhesive bonding of the pivot 245 and/or the hinge 260.

In this example, the temporary pacing lead 200 includes a Nitinol wire 255 within the wall structure or within a lumen of the lower member 220. In some examples, the nitinol wire 255 may be placed within a lumen of the upper member 215. In some examples, the nitinol wire 255 may be placed within both the upper member 215 and the lower member 220. The Nitinol wire 255 may, for example, assist in maintaining the axial length of the semi-circular curved outer surface 222 of the distal region 105 without plastic creep, as tension is placed on the respective loop fiber to bend the distal region 105 into a circular shape. For example, the Nitinol wire 255 may thereby aid in maintaining contact of the electrodes on the upper and lower members with the endocardial wall surface. The Nitinol wire 255 may, for example, advantageously permit the upper and lower members to return to a straight configuration when tension has been released from the loop fibers. In some examples, a wire (e.g., the Nitinol wire 255, other elastic material) may be chosen to advantageously increase a permitted bending within an elastic deformation region of the wire.

For example, a Nitinol wire may be located within the upper and/or lower member. The Nitinol wire 255 may, for example, be configured to aid the upper member 215 and/or the lower member 220 to maintain their rounded shapes when tension is applied to the upper loop fiber 230 and/or the lower loop fiber 235. In some examples, the nitinol wire 255 may be used to allow contact of the upper and lower members with opposing walls of the chamber of the heart with the upper member 215 and/or the lower member 220 without having a thermal creep effect on the upper member 215 and/or the lower member 220.

In some embodiments, a loop fiber for the upper member 215 and a loop fiber for the lower member 220 may, for example, extend separately to a manifold (not shown, e.g., the manifold 145) for placement of tension by an operator of the manifold. For example, the manifold may operate the loop fiber(s) using an actuator. The actuator may, for example, include a knob and/or handle (e.g., manual actuator). The actuator may, for example, include a motor and/or other powered actuator (e.g., having a mechanical and/or digital user interface). For example, some implementations may include an automatic actuator.

FIG. 2B depicts an embodiment of the temporary pacing lead including one loop fiber split into two separate loop fibers. As shown in FIG. 2B, a proximal loop fiber 225 splits into an upper loop fiber 230a and a lower loop fiber 235a (e.g., the upper loop fiber 230a and the lower loop fiber 235a may be separate wires joined to a third wire as the proximal loop fiber 225). In this example, the proximal loop fiber 225 is located in a proximal body 210a.

In some embodiments, the upper loop fiber 230 and the lower loop fiber 235 located near a flat surface 250 of the upper member 215 and/or the lower member 220 may induce the upper member 215 and/or the lower member 220 to compress the flat surface 250 and bend to form a circular shape at the distal region 105.

Several electrodes may, for example, be placed on both the upper and lower members. In the example shown in FIG. 2A, a first electrode 265a, a second electrode 265b, an third electrode 265c, and/or a forth electrode 265d may be attached to the upper member 215 and/or the lower member 220. For example, the electrodes 265a-d may be configured to make contact with the heart wall. The electrodes of the upper member and/or the electrodes of the lower member may, for example, be offset in an axial position. For example, the offset may be selected to provide a lowest profile for the distal region during delivery through the vasculature in a linear configuration.

FIG. 3A depicts a hinge when nonexpanded. A hinge 305 connects a lower member 310 and an upper member 315. The hinge 305 may include a metal material. The hinge 305 may include a polymeric material. The hinge 305 may include a composite material.

The hinge 305 may, for example, be configured as a ribbon. In some implementations, the hinge may, for example, be configured as a film. For example, the hinge 305 may include a thinned down region of a contiguous upper and lower member that is bent over at the hinge 305. The hinge 305 may, in some examples, be configured as a coupler device.

By way of example and not limitation, the hinge 305 may extend and bond into a lumen of the upper member 315 and/or lower member 310. The hinge 305 may, for example, be bonded to the flat surface of the lower member and/or upper member. The lower member 310 and/or the upper member 315 may be formed in a shape similar to a “D”. For example, in cross-section, a flat surface may be adjoined to a semi-circular curvature.

FIG. 3B depicts the hinge 305 of the embodiment from FIG. 3A. A flat hinge 305a is shown to connect to a lower member 310a and an upper member 315a in a distal region 105. A thermally formed thinned-down region 325 between the lower member 310a and the upper member 315a is shown to form the flat hinge 305a. The flat hinge 305a may, for example, be formed by thermally narrowing a portion of a catheter shaft. The flat hinge 305a may bend 330 to form a bent hinge 305b. The lower member 310a may bend 330 to form a lower member 310b. The upper member 315 may bend 330 to form an upper member 315b. The flat hinge 305a may bend 330 to form a bent hinge 305b. The bend 330 of the shaft of the upper member 315a and lower member 310a may form a distal tip 125 that makes contract with the myocardial walls of the heart.

FIG. 4A. depicts an extruded tubing 400 that forms an upper member 410 and a lower member 415 having a narrowed region 401. The narrowed region 401 may, for example, be thermally formed. A hinge 425 allows the narrowed region 401 to bend (e.g., the bend 330) more easily than other regions of the upper member 410 or the lower member 415 in a non-bent state. The hinge 425 may contain a ring or anchor that allows an optional attachment 445 with a loop fiber 405 in this example.

A loop fiber 405 may be attached via the optional attachment 445 to an anchor 420. The anchor 420, may for example, be a ring located at the narrowed region 401 of a distal region. By way of example and not limitation, the loop fiber 405 may extend within a loop fiber lumen 435 continuously through the upper member 410 located at a distal region distal region upper end 430 to lead to the lower member 415 located in a distal region lower end 440.

FIG. 4B depicts an extruded tubing that forms the upper and lower members having a thermally formed narrowed region in a bent state. The upper member 410a and lower member 415a of FIG. 4B may, for example, extend proximally with a D-shape cross-section throughout a proximal body 210 to a handle 450. The handle 450 may, for example, include the manifold 145 as described with reference to FIGS. 1A-1B. The upper member 410a and/or the lower member 415a may be D-shaped, with a flat surface 150 along the interior of its members. The flat surface 150, for example, may flexibly bond the upper member 410a and the lower member 415a. By way of example and limitation, the bonding may be formed with adhesives, thermal bonding, and/or solvent bonding. The loop fiber 405 may, for example, be attached with an optional attachment 445 to the anchor 420 located at the hinge 425 as shown in FIG. 4A.

As shown, a loop fiber 405c extends within a loop fiber lumen continuously from the lower member 415a end to a lead lower proximal end 455 in a distal region 105. A loop fiber 405d may extend within a loop fiber lumen continuously from the upper member 410a to lead to an upper proximal end 460 through the distal region 105. A loop fiber 405d may extend within a loop fiber lumen 435 (FIG. 4A) continuously from the lower member 415a end to a lead lower proximal end 455.

In some implementations, a loop fiber may, for example, extend continuously from the upper member to the lower member.

By way of example and not limitation, as shown in FIG. 4B, the loop fiber 405d located in the upper member 410a may, for example, be pulled independently from the loop fiber 405c located in the lower member 415a. In some examples, the loop fiber 405c and the loop fiber 405d located in each upper and lower member can be pulled with tension.

In some implementations, the loop fiber and/or pull wires may, for example, be configured to be pulled a specified (e.g., predetermined) distance to affect a curvature in the distal region 105 by a loop actuator 470. The loop actuator 470 may include a side member 475 (e.g., a spool). For example, the loop actuator 470 may include multiple spools. The spools may, for example, apply tension as they wind the loop fiber 405c, 405d and hold tension in the loop fiber 405c, 405d. The side member 475 may, for example, be released to release tension in the loop fiber 405c, 405d and allow the distal region of a lead body 480 to resume a linear shape.

A ribbon and/or wire 485 (e.g., Nitinol) may, for example, be placed on the upper member 410 and or the lower member 415 in the distal region 105 as shown in FIG. 4B to ensure that a rounded shape is given to the distal region 105 upon application of tension to the loop fibers by an actuator. The ribbon or Nitinol wire 485 may, for example, advantageously assist in return of the distal region to a linear shape upon removal of tension to the loop fibers or wires. The ribbon and/or wire 485 may, for example, advantageously provide the distal region 105 with a planar shape when it is formed in a circular shape upon application of tension to the loop fiber or wires.

During the assembly of the lead, as shown in FIG. 4A, electrode connecting wires 490a, 490b may be entered into the upper proximal end 460 and travel within a lumen in the proximal body 210 through the distal region lower end 440. One or both of the electrode connection wires 490a, 490b may, for example, include one or more electrodes. In some implementations, by way of example and not limitation, the electrode connecting wires 490a, 490b may each have a single electrodes (e.g., an electrode 495a and an electrode 495b). Some implementations may include more than two electrodes. For example, the distal region 105 may include four or more electrodes. The electrode connecting wires 490a may, for example, extend independently to the electrode 495a located in the distal region upper end 430.

By way of example and not limitation, the two electrode connecting wires may be entered into the lead lower proximal end 455 and extend independently to the two electrodes as partially shown in FIG. 4A. These two electrode connecting wires may, for example, be attached to an electrode in the distal region lower member 415a. The electrodes that are attached to the upper and lower members within the distal region may, for example, include a ring electrode having a D-shape that extends around the perimeter of the upper member or the lower member, for example. In some implementations, the electrodes can be located on the outer portion of the D-shaped upper member and/or lower member.

By way of example and not limitation, methods for forming the upper and/or lower member include multi-lumen extrusions (e.g., dual-lumen, tri-lumen). The extrusions may, for example, include a weak and/or thinned wall that can be easily separated to form multiple D-shaped portions of the distal region.

The distal region 105 can be bent at the narrowed region as shown in FIG. 4A. The upper member 410a and lower member 415a of the proximal body 210 may be bonded with the optional attachment 445 to form a lead body 480.

FIG. 4C depict an application of a tension mode of the extruded tubing 400 with a specified tension to the loop fiber 405c by a loop fiber control member (e.g., the loop actuator 470), which may cause the distal region 105 to form into a rounded shape. As shown, an expanded distal region 105 includes a smooth contour 465 for an exemplary distal tip 125. The smooth contour 465 may, for example, prevent accidental snagging of chordae tendineae or other structures within the heart or vascular.

A distal tip 125 may, for example, have a smooth contour. The smooth contour 465 may, for example, advantageously reduce or eliminate accidental snagging during an application of tension as shown in FIG. 4C. As depicted, the application of specified tension scenario involves a proximal body 210 that shapes the distal region 105 once the proximal body 210 is expanded. The proximal body 210 has an upper member and a lower member which are bonded by a bond 140 (e.g., a thermal bond, an adhesive bond). In this example, the upper member 410a and lower member 415a are coupled by the anchor 420b to form the smooth contour 465 for the distal tip 125. In some implementations, by way of example and not limitation, the diameter of the gap 170 (e.g., a circle) may include 2-2.5 cm±1 cm. For example, a length of the gap 170 between the bonded area and distal tip map may be calculated based on a circumference of the desired shape to contour the distal region.

FIGS. 5A-5B depict an alternative embodiment of a temporary pacing lead 500 in which the distal region is configured to form a circular shape. FIG. 5A depicts the temporary pacing lead 500 in an unexpanded state. A central loop fiber 505 is depicted in temporary pacing lead 500. The central loop fiber 505 may be attached to a hinge 510. The hinge 510 may connect an upper member 520 to a lower member 525 of the proximal body 210 of the pace lead 500. As depicted, a gap 170 is provided between the hinge and the proximate body where the upper and/or lower member couple via the hinge to the proximate body. The central loop fiber 505 may connect to a ring 540, anchor, and/or an optional attachment site.

In the depicted example, the central loop fiber 505 is located between the upper member 520 and lower member 525. The central loop fiber 505 is not located within a lumen of the upper member 520 or lower member 525. The central loop fiber 505 slidingly extends within a lumen of the proximal body 515.

As depicted, in some embodiments a flat sheet and 630 may be bonded to the flat surface of the upper member 520 and/or the lower member 525. The flat sheet may, for example, be configured to constrain the circular configuration to be maintained in a planar shape. The flat sheet may, by way of example and not limitation, include a polymer material. The flat sheet may, for example, include a metal material.

In some embodiments, the flat sheet 530 (e.g., a flexible but high tensile strength fiber) may be located within a body of the temporary pacing lead 500 to cause the loop to form a saddle shape that can follow the curvature of the endocardial surface of the right ventricle. In some implementations, for example, the loop may be operated into a saddle shape by orienting a flat sheet on the side of the lead body such that the saddle-shaped curve of the lead body corresponds with the short axis of a cross section of the flat sheet where the flat sheet prefers to bend.

In some implementations, for example, a flexible fiber placed on the inner curve of the lead body may, for example, allow the lead body to bend and form a convex curve on the side opposite to the fiber and form a lead loop curvature that matches the curve of a saddle and that of the right ventricle. An electrode 1 545 may be attached to a lower member 525. An electrode 2 550 may be attached to an upper member 520.

FIG. 5B depicts the temporary pacing lead 500 of FIG. 5A in an expanded state when tension is applied. A central loop fiber 505a is attached to a hinge 510a. The hinge 510a is attached to an upper member 520a and a lower member 525a. An application of tension to the central loop fiber 505a attached to the hinge 510a by the operator may cause the upper member 520a and the lower member 525a to bend to form a circular configuration maintained in a planar shape.

One or more embodiments and/or features described in FIGS. 1A-5B may be applied to the temporary pacing lead 500. In some embodiments, for example, the profile of the temporary pacing lead 500 of the previous embodiments may be small, for example, because the upper member 520a contains only half of the connecting wires and the lower member 525a may include half of the connecting wires.

FIGS. 6A-6C show additional features applicable to any of the embodiments shown in FIGS. 1A-5B. In some embodiments, an additional feature may include a fixation electrode located in a distal region of a temporary pacing lead to hold the distal region to the myocardial wall (e.g., a septal wall of the right ventricle near the apex of the heart at a location 1-2 cm (and/or ranging from 1-4 cm) from the apex of the heart).

FIG. 6A shows an exemplary temporary pacing lead 600 in a linear configuration that would be found during delivery of the exemplary temporary pacing lead 600 through the vasculature. For example, the linear configuration of the exemplary temporary pacing lead 600 may be found during removal of the exemplary temporary pacing lead 600 from the vasculature. The exemplary temporary pacing lead 600, in this example, includes a fixation electrode 605 that is enclosed within the distal region 105 of the exemplary temporary pacing lead 600. For example, the fixation electrode 605 may be contained within without extending outwards from an outer surface of the exemplary temporary pacing lead 600 in the linear delivery configuration.

In some implementations, the single fixation electrode 605 may be attached to a fixation electrode conduction wire 615. For example, the fixation electrode conduction wire 615 may extend proximally through a lead proximal body 620. For example, the fixation electrode conduction wire 615 may be attached to a fixation electrode slide member 625 located on the fixation electrode actuator 630 that forms a portion of a lead manifold 635 (e.g., a lead handle). Movement of the fixation electrode slide member 625, for example, by a physician or an operator may cause compression forces in the fixation electrode conduction wire 615 to be transmitted to the distal region 105. For example, the single fixation electrode 605 may be extended out of the distal region wall for a distance of 3 mm (e.g., 2-5 mm) and extend into the myocardial wall (as shown, for example, in FIG. 6C).

In some implementations, for example, application of energy to the fixation electrode 605 via an electrode connector 640 located at the lead manifold 635 can assist in allowing the fixation electrode 605 to penetrate more easily into the myocardial tissue. For example, the energy may be thermal energy. In some implementations, a tip of the fixation electrode 605 may include a heating element. The heating element may, for example, include a resistive element (e.g., nichrome wire).

In some implementations, the energy may include radiofrequency energy (RF energy), for example. In some embodiments using an RF generator (e.g., commonly used in medical procedures such as crossing the atrial septum, for example) connected to the electrode connector that extends to the fixation electrode, for example, a counter electrode placed onto the patient's body surface can be used to provide a closed pathway for current supplied by the RF generator.

In the depicted example, the fixation electrode serves to hold the distal region 105 of the exemplary temporary pacing lead 600 in a fixed position relative to the myocardium. In the depicted example, the fixation electrode 605 is also configured and operated to function as an electrode. For example, the fixation electrode may be configured to provide sensing and/or activation signals to the myocardium, in some embodiments.

In some implementations, the fixation electrode 605 may be chosen as one of an electrode pair for pacing the patient based on the electrode pair having a high sensing voltage from the myocardium and a low threshold current for pacing the myocardium in comparison to other electrode pairs.

FIG. 6B shows an exemplary distal region 105 of the exemplary temporary pacing lead 600 of FIG. 6A after forming a loop 650. For example, the loop 650 may be formed after the loop control member 110 has been placed into tension as described in the embodiments described with reference to FIGS. 1A-5B. The loop control member 110 extends from an anchor 645 (e.g., a ring, hinge, or other member) located at or near a distal tip 655 through a separate lumen, for example, within the temporary pacing lead 600 and through the lead proximal body 620. For example, the distal region 105 may be attached to a loop slide member 660 located on a loop actuator 665 of the lead manifold 635 as described with reference to FIG. 6A. The loop control member 110 can be a metal or polymer wire or fiber.

In some implementations, the loop control member 110 may include a ribbon having a rectangular cross-section.

In some embodiments, the loop control member 110 may include both tensile and compressive strength to activate the distal region 105 into a loop in an expanded mode and/or place the distal region 105 into a linear shape in a linear mode.

For example, an operator can place the loop control member 110 under tension to cause the distal region 105 to form a loop as shown in FIG. 6B. The exemplary temporary pacing lead 600 can be placed within the right ventricle, for example, with this configuration. For example, the distal region tip 655 may have a round shape to advantageously safely contact the apical region of the ventricle without concern for potential perforation of the myocardial wall near the apex, for example. The fixation electrode may, for example, be enclosed within the wall of the exemplary temporary pacing lead 600 and may not extend out of an outer surface of the distal region 105 as shown in FIG. 6B.

After the exemplary temporary pacing lead 600 has been positioned within the body cavity (e.g., right ventricle), for example, the fixation electrode 605 can be advanced outwards of the lead outer surface through an opening in a lead wall 675 to place the fixation electrode 605 into a septal wall 670 of the heart, for example, as shown in FIG. 6C. The operator places the fixation electrode conduction wire 615 under compression as the operator activates the fixation electrode slide member 625 located on the fixation electrode actuator 630 to advance the fixation electrode 605. The fixation electrode 605 extends outwards from a lead distal body opening 680 in a direction radially outwards from the loop 650 formed in the distal region 105 and with; an angle directed away from a heart apex 685 as shown in FIG. 6C. With the loop 650 extending between the septal wall 670 and the lateral wall 690 of the heart, the fixation electrode 605 is most effectively positioned along the exemplary temporary pacing lead 600 such that the fixation electrode 605 extends radially outwards from the loop 650. In some embodiments, the loop 650 may be rotated into a position within the right ventricle such that the loop plane lies more parallel with the lateral wall 690 and septal wall 670, the positioning of the lead distal body opening 680 for passage of the fixation electrode 605 can be located on the side of exemplary temporary pacing lead 600 and the fixation electrode 605 is directed during activation outward into the tissues in a direction that is more perpendicular to the plane of the loop 650.

The fixation electrode slide member 625 can also be formed to transmit a rotational torque to remove or screw a coiled fixation electrode rather than the needle shaped curved electrode shown in FIGS. 6A-6C. The exemplary temporary pacing lead 600 can be easily removed by placing the fixation electrode conduction wire 615 under tension to remove the (linear or curved) fixation electrode 605 from the myocardium as shown in FIG. 6C with the fixation electrode 605 extending at an angle away from the heart apex 685. The loop actuator 665 can then be activated by the operator to apply compression as needed to the loop control member 110 to allow or cause the distal region to form a linear shape for removal of the lead from the heart and vasculature of the patient.

The fixation electrode 605 can be formed from an electrically conducting metal (e.g., platinum-iridium, other alloys of platinum, other metal used in pacemaker leads). For example, the fixation electrode 605 can be formed from stainless steel. For example, a pacing current can be increased without concern for affecting battery life of an implanted pacing pulse generator. The fixation electrode 605 can be formed with a sintered metal surface to enhance the surface area for current flow from the electrode to the myocardial tissue.

Although FIG. 6A shows only one fixation electrode, it is understood that more than one fixation electrode can be located along the lead distal region that is in contact with either the septal wall, the lateral wall or both walls of the right ventricle, for example.

Following activation of the fixation electrode into the myocardium, the physician operator can then further rotate, advance, or move the pacing lead handle manifold to provide contact of other electrodes such as ring electrodes (e.g., the first electrode 165a), for example, located on the lateral wall 690 of the right ventricle, for example, or into contact with the wall of the right ventricle to obtain improved contact of one or more electrodes with the myocardial wall.

As shown in FIG. 6A the electrode connecting wires 160 and the fixation electrode conduction wire fixation electrode conduction wire 615 may each end in an electrode connector located at the lead manifold 635. FIG. 6A shows an example of a lead having three electrode connectors, two of which will be used by the physician to form the optimal electrode pair that is used to pace the heart. In some embodiments, a lead (e.g., any of the temporary pacing leads described with reference to FIGS. 1A-6C) may include more than three electrodes located in the distal region of the pacing lead. The physician can choose two electrode connectors to attach via an extension cable to the anode receptacle and cathode receptacle of the pulse generator and evaluate the sensing voltage and the current need to provide capture of the myocardium. The physician can then choose an alternate pair of electrode connectors and identify the electrode connector pair that provides the highest sensing voltage and the lowest current needed to provide capture.

FIGS. 7A-7F show examples of fixation electrodes that can be used with one or more embodiments. FIG. 7A shows a fixation electrode 700 formed from a curved element (e.g., wire) located at the end of a fixation electrode conduction wire 705. The fixation electrode 700 may, for example, be contiguous with the fixation electrode conduction wire 705. In some examples, the fixation electrode 700 may be joined (e.g., by a metal joining process) to the fixation electrode conduction wire 705. The fixation electrode 700 and the fixation electrode conduction wire 705 can be formed from electrically conducting metals. Metals may, for example, include metals used throughout the pacing lead industry. Metals may, for example, include platinum alloys and/or stainless steel. The fixation electrode 700, in this example, includes a sintered conducting metal surface 710. The sintered surface may, for example, advantageously increase surface area for contact with the myocardial tissues. For example, the sintered surface may advantageously increase friction and/or increase pullout strength.

As an illustrative example, the length of the fixation electrode 700 that extends into the myocardial wall tissue may be 2-5 mm. In some implementations, for example, the fixation electrode may be 3 mm.

In some implementations, the fixation electrode conduction wire 705 may, for example, be configured to apply compression and/or tension to activate the fixation electrode 700 out of the lead, and/or to pull the fixation electrode 700 back into a lead body (e.g., the distal region 105). In some implementations, the fixation electrode conduction wire 705 may be insulated. For example, the fixation electrode conduction wire 705 may travel within a lumen through the lead body to the lead manifold (e.g., the lead manifold 635). The wire 705 may be attached (e.g., in the lead manifold 635) to the fixation electrode slide member 625 of the fixation electrode actuator 630. The actuator 630 may, for example, be manipulated by the operator to advance and/or withdraw the fixation electrode 700 relative to the distal region 105.

As an illustrative example shown in FIG. 6A, a fixation electrode conduction wire 705 may extend to an electrode connector 695 located on the lead manifold 635. In this example, the electrode connector 695 is connectable to an extension cable 696 that places the electrode connector 695 in electrical contact with the anode or cathode pole of a pulse generator 610. For example, the physician can choose which electrode connector pair provides the best electrode pair for pacing with the best capture.

FIG. 7B shows a bifurcated fixation electrode 715 where two fixation electrodes (e.g., generally curved in this example) are attached to a fixation electrode wire 720. The structure of each branch of the fixation electrode 715 may, for example, be implemented as described for the fixation electrode 700. Advancement of the fixation electrode wire 720 may, for example, cause both branches of the bifurcated fixation electrode 715 to advance out of the lead body opening 725 and extend into the myocardial wall tissue. The electrode 715 may, for example, engage with the tissue in bifurcated directions. The bifurcated directions may, for example, advantageously increase holding power. The increased holding power may, for example, advantageously prevent lead migration. The multi-engagement fixation electrode 715 may, for example, enhance surface area for contact with the myocardial tissues. The additional surface area may, for example, advantageously provide improved electrical conduction.

FIG. 7C shows two fixation electrodes 730. The electrodes 730 may, be contiguous with a fixation electrode wire 735. The electrodes 730 may, for example, be attached to the wire 735.

As depicted, the wire 735 connects to a fixation electrode slide member located on the lead manifold. In this example, each fixation electrode 730 extends out of a separate opening in the outer surface of the lead distal region. The openings in the surface of the lead may, for example, be spaced apart by a predetermined distance (e.g., equal, linearly varying, monotonically varying). In some examples, the predetermined distance may be between 0.5-2 cm along the axial length of the distal lead body. In some examples, the predetermined distance may be 1 cm.

In some embodiments, the two fixation electrodes 730 may provide a single electrode signal for an electrode pair. The electrode pair may, for example, utilize yet another electrode located elsewhere along the distal lead body (e.g., a ring electrode).

FIG. 7D shows two fixation electrodes 740. The fixation electrodes 740 may, for example, be implemented as disclosed at least with reference to the fixation electrode 700 described in FIG. 7A. Each of the two fixation electrodes 740 may, for example, be attached or contiguous with a separate fixation electrode conduction wire 745. Each fixation electrode wire 745 is attached to a separate fixation electrode slide member located on the actuator, for example.

One or both of the fixation electrodes 740 can be located to be activated into tissue. For example, the electrodes 740 may be located to engage the septal wall and/or the lateral wall of the right ventricle.

In some implementations, the direction of activation of the fixation electrode is directed to extend from the opening in the lead distal body at an angle away from the apex of the heart. The angle may, for example, advantageously resist or prevent migration of the distal body loop away more efficiently from the apex of the heart. For example, if a first fixation electrode is positioned on the septal wall of the right ventricle and a second fixation electrode is positioned on the lateral wall of the right ventricle, then the fixation electrode conduction wire of the first fixation electrode conduction wire may be placed under compression to advance the first fixation electrode outwards into the myocardial tissues and the second fixation conduction wire could be placed under tension to advance the second fixation electrode outwards.

FIG. 7E shows a fixation electrode 750 that is activated out of the distal body opening by applying tension to the fixation electrode conduction wire 755. In some embodiments, a fixation electrode located in the lower portion of the distal region (e.g., as shown in FIG. 6A) may include a fixation electrode, for example, that is activated outwards into the myocardial tissue via application of In some examples, tension to the fixation electrode conduction wire. In some implementations, the fixation electrode 750 could be retracted back into the distal body opening by application of compression to the fixation electrode conduction wire 755.

FIG. 7F shows a screw fixation electrode 760. For example, the screw fixation electrode 760 may be a braided structure formed from multiple metal relatively smaller metal fibers (e.g., stainless steel) to provide for ease of bending while allowing for torque transmission from the fixation electrode actuator to the screw fixation electrode to turn the screw fixation electrode 760 and enter the myocardial tissues. A screw fixation electrode wire 765, in some implementations, may advance axially as it turns to allow for axial and rotational movement of the screw fixation electrode 760 as it enters into the myocardial tissue. In some examples, the axial and rotational movement may be supplied simultaneously by movement of the fixation electrode slide member by the operator.

In some implementations, the axial length of the screw fixation electrode that extends into the myocardial tissues may range between 2-5 mm. In some implementations, for example, the length may be 3 mm.

In some embodiments, the screw fixation electrode may, for example, include a sintered metal outer surface. The sintered surface may, for example, provide additional surface area for contact with myocardial tissue.

In some implementations, for example, removal of the screw fixation electrode may be accomplished following the pacing procedure by reversing the rotational movement used to advance the screw fixation electrode via the fixation electrode slide member.

FIGS. 8A and 8B show an exemplary lead body 800 in an expanded configuration located in the right ventricle of the heart. In FIG. 8A, a fixation electrode 805 is shown extending into the septal wall 670 at a location of 1 cm (range 0.5-2 cm) from the apex of the heart. Two electrodes 810 (e.g., ring electrodes) are provided. As depicted, the electrodes are located adjacent the lateral wall 690. The electrodes may, for example, be positioned at a location of 0.5-3 cm (e.g., 1 cm) from the apex of the heart.

In some embodiments, the exemplary lead body 800 can be rotated, withdrawn, and/or advanced (e.g., slightly), such as to improve the positioning of one or more of the electrodes 810 into contact with the lateral wall 690, for example, such as shown in FIG. 8A. The axial distance along the lead body 800 from the fixation electrode 805 to one or both of the ring electrodes 810 may be 1-5 cm (e.g., 2 cm).

In the depicted example, the proximal lead body 815 extends through an introducer 820 (e.g., as sheath) located in the inferior vena cava (IVC). The proximal end of the introducer 820 is releasably attached to a Tuohy Borst device 830 that is configured to provide friction relative to the proximal lead body 815. The Tuohy Borst device 830 is activated to apply frictional holding to the lead body 800 after the lead body 800 is positioned within the right ventricle, for example, and forward pressure is placed onto the lead body 800. The lead body 800 is placed into a compressive mode such that the distal loop 825 of the lead is placed and held into contact with the myocardial tissues near the apex of the heart. The proximal lead body 815 may, for example, thereby be held in place such that movement of the proximal lead body 815 is resisted axially and/or radially relative to the IVC. For example, the position of the distal loop 825 may be unable to move away from the apex of the heart and/or unable to move rotationally within the right ventricle. This fixing of the position of the distal loop 825 may, for example, advantageously maintain a current capture threshold once the target (e.g., optimal) electrode pair is identified and the Tuohy Borst device 830 is secured to the proximal lead body 815. For example, fixing the position of the distal loop 825 may advantageously prevent electrical contract from being interrupted due to patient movement and/or due to movement associated with the beating of the heart.

In some examples, one or more fixation electrodes may be provided as active fixation elements. For example, in some embodiments, the fixation electrodes described with reference to FIGS. 7A-F) to physically hold the distal loop 825 to the septal or lateral wall of the right ventricle may be applied as described in FIGS. 6A-6C.

FIG. 8B shows an extended introducer 850 that extends further into the IVC. For example, as shown, the extended introducer 850 may extend to a location near or extending into the tricuspid valve. The proximal end of the extended introducer 850 may, for example, be reversibly attached to a Tuohy Borst device 830 that fixes the movement axially and/or rotationally of the proximal lead body 815 to the extended introducer 850. The extended length of the extended introducer 850 along with the Tuohy Borst device 830 maintains the distal loop 825 of the pacing lead in a fixed position within the right ventricle. The distal loop 825 may for example, be anchored such that movement in a direction away from the apex of the heart is resisted. For example, support of the proximal lead body 815 against movement may be provided via a tight fit between the proximal lead body 815 and the extended introducer 850 (spacing of 0.003 inches, range 0.002-0.004 inches) and/or a small distance of about 10-12 cm from the distal loop 825 to a distal end of the extended introducer 850 (e.g., fixed from axial and/or rotational movement).

FIGS. 9A-9G show illustrative methods of assembling an exemplary lead body 900 (e.g., the temporary pacing leads described with reference to FIGS. 1A-8B). Other methods of assembly are contemplated. FIG. 9A shows a distal region of a lead formed from a multi-lumen extrusion in a linear form. The depicted embodiment has two fixation electrodes 905. The fixation electrodes 905 may be configured to both extend outwards at an angle away from the heart apex. The fixation electrodes 905 may, for example, be activated into the myocardial tissue by applying compression to a fixation electrode conduction wire 910. The fixation electrodes 905 may be withdrawn from the tissues by applying tension to the fixation electrode conduction wire 910. Application of tension to withdraw the fixation electrodes 905 may, for example, advantageously provide consistency in removal, such as due to the ability of the fixation electrode conduction wire 910 to apply a higher tensile force than compressive force.

In the depicted example, the fixation electrode conduction wires 910 extend across a hinge region 915 and are attached to a first and a second fixation electrode actuators located on a manifold (e.g., the lead manifold 635). The loop control member 110 extends through the lead body 900 to a loop actuator located on the lead manifold. One or more electrodes 930 (e.g., ring electrodes, as shown) are placed, in the depicted example, on the upper portion 920 and lower portion 925 with separate conduction wires extending to the lead manifold. After the fixation electrodes, the fixation electrode conduction wires, the loop control member, ring electrodes, and other conduction wires have been placed on and within the lead body 900, the lead body may, for example, be bent at the hinge region 915 as shown in FIG. 9B. The upper portion 920 of a lead distal region 935 and the lower portion 925 may, for example, be configured to form a loop in the distal region 935 upon activation of the loop control member 110. The upper portion 920 and the lower portion 925 may, for example, then be attached to a manifold.

FIGS. 9C and 9D show a pacing lead manufacturing method. The method may, for example, be implemented such as disclosed at least with reference to FIGS. 9A and 9B, except that a fixation electrode 950 in FIGS. 9C-D may be activated outwards into the myocardial tissues via application of tension to a fixation electrode conduction wire 955. In some examples, the fixation electrode 950 may be removed via application of compression to the fixation electrode conduction wire 955. The fixation electrode conduction wire 955 may, for example, not extend past the hinge region but may, for example, extend directly toward the manifold. Other aspects of this embodiment including the loop control member, ring electrodes, and other conduction wires may be implemented such as disclosed at least with reference to FIGS. 9A and 9B.

FIGS. 9E-9G show an illustrative method of assembly in which the upper portion lead body of the distal region is beveled. As seen in FIG. 9E, the distal region 105 includes a fixation electrode 940 and one electrode 945 (e.g., ring electrode, as shown). For example, more than one of each type of electrode can be found in either or both the upper portion or lower portion of the distal region 105. In this example, the fixation electrode 940 is found in the upper portion 920 near a bevel 960 and extending past the hinge region 915 to allow withdrawal of the fixation electrode 940 via application of tension to the fixation electrode conduction wire 910 via a fixation actuator as shown in FIG. 9F.

In some implementations, a bevel tip 965 of the distal region 105 may be bonded permanently to the lead proximal body 620 in a manner that allows for fixation of a bevel attachment 970. Such bonding methods may include, by way of example and not limitation, adhesives, solvent bonding, thermal bonding, and/or other bonding methods found in the medical device industry. One or more ring electrodes 975, for example, may be placed in the upper or lower portions or both and each ring electrode is attached to an insulated conduction wire that extends in a direction towards the lead manifold as shown in FIG. 9G. The loop control member 110 is attached to the lead body near the hinge region 915 via a loop attachment 980. For example, the loop attachment 980 may include an anchor, a ring, and/or other non-moveable member of the lead body near the hinge region 915. The loop control member 110 extends toward the lead manifold 635 as shown in FIG. 9G and is attached to the loop actuator 665. The distal loop 825 may, for example, be formed by applying tension by the operator to the loop control member 110 as shown in FIG. 9G. The loop actuator 665 may, for example, then hold the tension to maintain the loop in its curved shape.

To alter the shape of the loop to a smaller diameter loop, for example, the loop actuator 665 may be manipulated by the operator to apply compression to the loop control member 110. Further application of compression to the loop control member 110 may, for example, induce the loop to take a linear shape for removal. In some examples, the upper and/or lower portion of the distal region 105 may contain an elastic member having a linear equilibrium shape. The elastic member may, for example, cause the loop to assume a linear shape upon removal of the tension that is being held by the loop actuator 665.

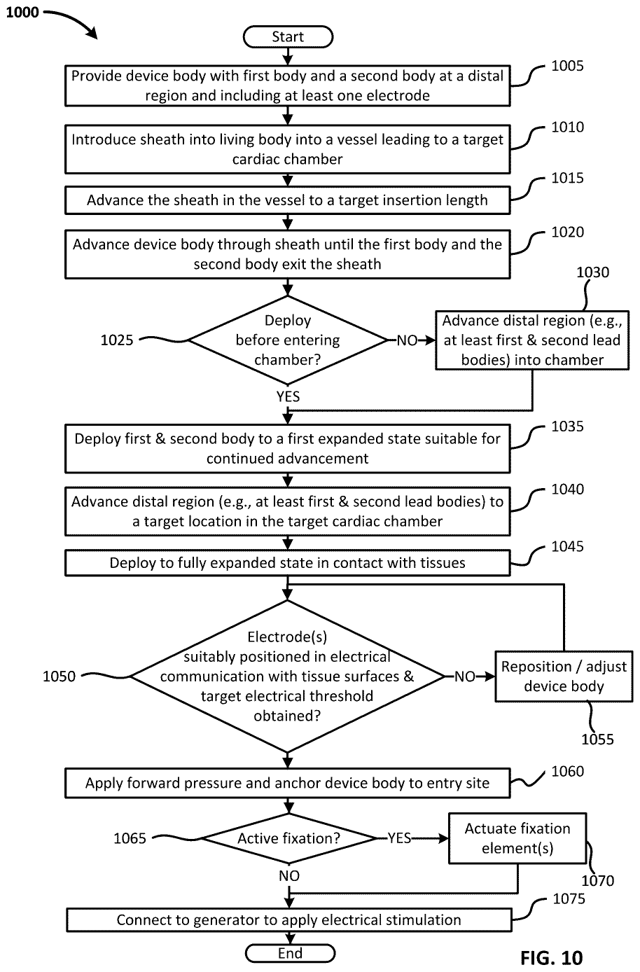

FIG. 10 depicts an illustrative method 1000 of temporary stimulation using, for example, any of the embodiments disclosed at least with reference to FIGS. 1A-9G. In a step 1005, a device body is provided. The device body may, for example, include a first body and a second body at a distal region. The distal region may include at least one electrode coupled to an electrode conduction wire extended through a proximal end of the device body.

A sheath is introduced, at a step 1010 into a living body (e.g., at a groin entry point, at a neck entry point). The sheath may be introduced into a vessel leading to a target cardiac chamber (e.g., the right ventricle). In a step 1015, the sheath is advanced in the vessel to a target insertion length (e.g., as disclosed at least with reference to FIGS. 8A-8B). The device body is advanced, in a step 1020, through the sheath until the expanding distal end (e.g., at least the first body and the second body) exit the sheath.

If it is determined, in a decision point 1025, not to at least partially deploy the expanding distal end before entering the target chamber, then the distal region (e.g., at least the first and second body) is advanced into the target cardiac chamber in a step 1030.

If it is determined, in the decision point 1025, to at least partially deploy prior to entering the target chamber (e.g., to operate into a partially expanded state, such as to reduce a risk of puncture of tissue), or after step 1030 is completed, then the expanding distal end (e.g., at least the first and second bodies) are deployed to a first expanded state (e.g., partially expanded) suitable for continued advancement (e.g., partially expanded without applying excessive pressure to a vessel wall and/or valve aperture) in a step 1035. The distal region is then advanced to a target location (e.g., an apex of the right ventricle) in the target cardiac chamber in a step 1040.

In a step 1045, the expanding distal end (e.g., the first body and the second body) are expanded to a second expanded state (e.g., fully expanded) such that the first body and the second body contact different tissue surfaces (e.g., septum, ventricle wall, opposing myocardial surfaces) within the target cardiac chamber.

In a decision point 1050, it is determined if the electrode(s) are suitably positioned in electrical communication with the tissue surfaces. For example, as depicted, suitable positioning may be determined by whether a target electrical threshold is obtained. In some implementations, for example, suitable positioning may be at least partially determined by imaging (e.g., radiography, ultrasound). In some implementations, multiple conductors (e.g., conductor pair combinations) may be tested (e.g., manually, automatically such as by an automatic switch box) to determine if any electrodes are in suitable contact.

If it is determined in the decision point 1050 that the at least one electrode is not suitably positioned, then the device body is repositioned and/or adjusted (e.g., expanded, contracted, rotated, re-shaped such as by differentially expanding/contracting the first and second lead bodies) in a step 1055, and then the method returns to the decision point 1050.

In the method 1000, the device body is anchored (e.g., ‘passively fixated’) in a desired position (e.g., after decision point 1050 as shown, before decision point 1050 such as after step 1045) by applying forward pressure to the device body. In some embodiments forward pressure may be maintained, such in the example disclosed in FIG. 10, by anchoring (e.g., suturing) the device body to the entry site of the living body. The device body may, for example, advantageously be anchored thereby in the target cardiac chamber by the forward pressure. In some examples, a Tuohy Borst device may be positioned near the introducer manifold may. The Tuohy Borst device may, for example, be used to frictionally hold the proximal lead body to the introducer and/or maintain the forward pressure of the device body into the cardiac chamber.

If it is determined, in a decision point 1065, to use active fixation (e.g., in addition to the forward pressure, in replacement of the forward pressure), then at least one fixation element is actuated in a step 1070 (e.g., as disclosed at least with reference to FIGS. 6A-9G). In some implementations, deploying the active fixation element may include activating (e.g., temporarily) an energy-assisted anchoring initiation element (e.g., a heating element, an RF element).

In a step 1075 (e.g., after step 1070 or decision point 1065, as shown, previous to decision point 1065 such as after step 1040) the electrode(s) are electrically connected to a pulse generator, such as to apply electrical stimulation (e.g., temporary pacing).

Although exemplary embodiments have been described with reference to FIGS. 1A-9G, other implementations may be deployed in other industrial, scientific, medical, commercial, and/or residential applications.

In some implementations, the loop 650 may advantageously provide an atraumatic way of advancing the distal region 105 within a body cavity. For example, a round shape loop may, for example, be highly unlikely to perforate and/or tear body tissues while delivering. For example, a broad loop may advantageously prevent perforation of a body tissue (e.g., the apex). In some implementations, the distal region 105 may include multiple conductor contacts. For example, the distal region 105 may include two or more leads fixation electrode 605.

In some implementations, the manifold 145 may include a switch box. For example, the switch box may include selective coupling to two or more electrodes. For example, the electrodes may include standard connecting cable with anode and cathode (e.g., re-sterilizable). For example, the manifold 145 may include a pacemaker box connects to one or more of the electrodes. In some examples, the manifold 145 may omit a switch box. For example, the electrodes may be manually connected and/or connected by a plug assembly. The electrodes may, for example, be connected directly to a generator. Embodiments without a switch box may, for example, advantageously reduce cost.

In some implementations passive fixation may include, for example, the selectively expanded upper and lower member. The passive fixation may advantageously maintain an excellent and firm contact (e.g., electrical contact) with tissue (e.g., a heart wall) when the distal region 105 is opened at a predetermined location (e.g., the apex of the right ventricle of the heart). In some examples, for example, the manifold 145 may be configured to maintain forward pressure on the loop control member 110.

In some implementations, the fixation electrode 605 may advantageously provide improved migration resistance of the distal region 105. In some implementations, the fixation electrode 605 may include a hook. For example, the temporary pacing lead 100 may include two independent actuators. For example, one actuator may be used to deploy the upper and lower members into a loop. For example, one actuator may be used to deploy the hook. For example, the loop may be configured to be expanded in artery.

In some implementations, the fixation electrode 605 may be configured to extend, while activated, outwards from the lead body opening 725 at a predetermined angle. For example, the safest place to deploy anchor is into septum which will be parallel to the flat plane the loop makes.

In some implementations, the loop may include a saddle and/or a cylinder shape in some plane (e.g., to match shape of septum). In some implementations, the loop control member 110 may be configured to control the expanded shape at the distal region 105.

In some implementations, a fixation element (e.g., fixation electrode) may be configured to deploy automatically. For example, the fixation element may be biased such that the fixation electrode automatically extends from the lead body when the lead body is operated into a specific shape and/or level of expansion. Such embodiments may, for example, advantageously reduce cost and/or operating complexity.

Some embodiments may include sensors to detect deployment of the lead body. For example, some embodiments may be provided with sensors at one or more locations along an upper and/or lower lead body. For example, a strain gage may advantageously provide a metric of a level of flexing and/or a shape of the loop. In some examples, an inertial measurement unit (IMU), accelerometer, and/or gyroscope may be configured, for example, to measure orientation. In some examples, the lead body may, for example, include an optical sensor (e.g., camera) configured to provide visualization of placement. In some examples, the lead body may include proximity and/or contact detection. In some examples, the lead body may include pressure and/or force sensors (e.g., on an outside surface of the upper and/or lower lead bodies), such as configured to measure engagement force (e.g., with the tissue).

In some implementations, an outer radius of the loop may include a pre-formed fiber (e.g., flexible but not elastic). For example, an inner curve of the loop may include a stiff member to advantageously prevent the inner curve of the catheter from stretching, while the outside curve stretches to conform to anatomy. In some implementations, a tip of the anchor 420b may be electrically activated with RF to facilitate engagement with cardiac tissue to minimize amount of force to initiate penetration into myocardium.

Although various embodiments are disclosed with reference to temporary cardiac pacing, other implementations are possible. In some examples, the lead body may be used in any cavity in the body.

For example, in some embodiments, a lead body (e.g., the lead body 800) may be configured and/or used in neurostimulation.

For example, the temporary pacing lead 100 may be used for stimulating stomach and/or gastrointestinal (GI) regions.

In some implementations, the temporary pacing lead 100 may be used in urology. For example, the distal region 105 may be configured to expand in the bladder, urethra, and/or ureter. For example, the temporary pacing lead 100 may be used to prevent spasming, such as after stone removal.

In some implementations, the lead body (e.g., of the temporary pacing lead 100, the lead body 800) may be implemented as a deployment platform. For example, the lead body may be configured as a selectively deployable temporary deployment platform. The lead body may be provided with one or more sensors (e.g., temperature sensors, pH sensors, camera) and/or actuators (e.g., thermal application devices, electrical application devices). For example, the expanding lead body may serve as a temporary, non-occlusive anchor. For example, the expanding lead body and/or fixation anchor (e.g., as an electrode, as a non-electrode) may be implemented as a temporarily anchored delivery and/or monitoring platform.

In some implementations, for example, a physician may operate the lead body into a desired region, and deploy the lead body. The lead body may be anchored in place, for example, by shape and/or by deployment of one or more fixation anchors. In some implementations, for example, one or more effectors (e.g., biopsy tool, surgery tool) and/or sensors (e.g., cameras, analyte detectors, force sensors) may be operated from the platform. In some implementations, one or more effectors may be used for ablation therapy in one or more regions of the body.

Although embodiments disclosed herein are disclosed with an upper body (e.g., a first body) and a lower body (e.g., a second body), other embodiments are possible. For example, more than two lead bodies may be provided. For example, three lead bodies may advantageously provide increased contact in a three-dimensional cavity (e.g., ventricle). In some implementations, more than three lead bodies may be provided.

Some embodiments may, for example, be implemented such as disclosed at least with reference to U.S. Application Publication No. 20220118261 (referred to as the '261 publication), which is a publication of U.S. application Ser. No. 17/075,409, titled “Curled Shaft Temporary Pacing Lead,” filed Oct. 20, 2020 by Wesley Robert Pedersen, et al., the entire contents of which are incorporated by reference. For example, some embodiments may include an integrated and/or fully implantable pulse generator (e.g., such as disclosed at least with reference to [0027] of the '261 publication). Some implementations may, for example, include structures (e.g., lead-manifolds, switch boxes, pulse generators, electrode(s), sheaths/introducers) and/or methods (e.g., implantation, placement, threshold measurement) such as disclosed at least with reference to FIGS. 1A-30B).

In an illustrative aspect, a temporary intra-cardiac pacing device (TICPD) may include a device body (600). The device body may include a first body (135) and a second body (130) at a distal region (105). The distal region may include an electrode (605, 810) coupled to an electrode conduction wire (705) extended through a proximal end (620) of the device body. The electrode is configured to contact a wall tissue and electrically connect the wall tissue to a pulse generator (610). The TICPD may include a coupling system including a loop control member (110) and extending longitudinally within the device body, and disposed between the first body and the second body. The TICPD may include a loop actuator (630) disposed at the proximal end of the device body and coupled to the loop control member. The TICPD may be configured such that, when the loop control member is activated by tension applied from the loop actuator, the coupling system operates the first body and the second body to separate to form a loop in the distal region and to bring the electrode into contact with the wall tissue.

The electrode may include a retractable fixation electrode. The electrode conduction wire may include a fixation electrode conduction wire coupled to a slide member at the proximal end of the device body. The retractable fixation electrode may be disposed at a lead distal opening at the distal region. The TICPD may be configured such that activation of the slide member causes the retractable fixation electrode to extend in a predetermined angle from the loop away from a distal end at the lead distal opening, such that the retractable fixation electrode is releasably coupled to the wall tissue.

The TICPD may further include multiple electrodes.

The electrode of the TICPD may include at least one ring electrode.

The retractable fixation electrode may include a coiled end. The slide member may be configured to transmit a rotational torque to cause the coil end of the retractable fixation electrode to screw into the wall tissue.

The fixation electrode conduction wire may be connected to two or more retractable fixation electrodes. Each of the retractable fixation electrodes may extend out of separate lead distal openings at the distal region.

The loop may include a planar gap. The planar gap may include an end-to-end separation distance between 2-2.5 cm.

The coupling system may include a hinge disposed near the distal region of the device body and coupled to the loop control member such that activation of the loop control member causes the hinge to be pulled and causes a bended expansion of the first body and the second body.

The coupling system may include a hinge thermally bonded at the distal region of the device body.

The coupling system may include a pivot adjoining a proximal end of the first body and the second body, such that activation of the loop control member causes the first body and the second body to separate at the pivot.

The distal region may include a beveled portion, such that the loop is configured to form a curved shape via the loop control member.

In an illustrative aspect, an in vivo deployment platform (IVDP) may include a device body (600). The device body may include a first body (135) and a second body (130) at a distal region. The distal region may include an end node (605, 810) coupled to a conductor (705) extended through a proximal end (620) of the device body. The end node may be configured to contact a wall tissue of a body cavity. The IVDP may include a coupling system including a loop control member (110) and extending longitudinally within the device body, and disposed between the first body and the second body. The IVDP may include a loop actuator (630) disposed at the proximal end of the device body and coupled to the loop control member. The IVDP may be configured such that, when the loop control member is activated by a tension applied from the loop actuator, the coupling system induces the first body and the second body to separate to form a loop at the distal region, wherein the loop is held by the applied tension, such that, when the distal region is inserted into the body cavity and the loop control member is activated, the loop at the distal region of the device body travels within the body cavity without damaging wall tissues of the body cavity.

The end node may include a sensor. The sensor may include a camera.

The end node may include an actuator. The actuator may include a thermal application device.

The end node may include a conducting ring.

The end node may include a retractable fixation element coupled to a fixation wire. The fixation wire may be coupled to a slide member at the proximal end of the device body. The retractable fixation element may be disposed at a lead distal opening at the distal region, such that activation of the slide member causes the retractable fixation element to extend in a predetermined angle outwards from the loop at the lead distal opening, such that the retractable fixation element is releasably coupled to the wall tissue. The retractable fixation element may include an electrode.

The retractable fixation element may include a coiled end. The slide member may be configured to transmit a rotational torque to cause the coil end of the retractable fixation element to screw into the wall tissue.

The fixation wire may be connected to at least a second retractable fixation element. Each of the retractable fixation elements may extend out of separate lead distal openings at the distal region.

The distal region may include a beveled portion, such that the loop is configured to form a curved shape via the loop control member.

The coupling system may include a hinge disposed near the distal region of the device body and coupled to the loop control member, wherein activation of the loop control member causes the hinge to be pulled and causes a bended expansion of the first body and the second body.

For example, one or more elements of the temporary intra-cardiac pacing device of any of [0149-159] may be combined with one or more elements of the in vivo deployment platform of any of [0160-168]. For example, one or more elements of the in vivo deployment platform of any of [0160-168] may be combined with one or more elements of the temporary intra-cardiac pacing device of any of [0149-159]. For example, in some embodiments one or more elements of any of [0149-168] may be manufactured, configured, and/or operated according to any method disclosed herein including, by way of example and not limitation, any of steps 1005-1075 of method 1000.

A number of implementations have been described. Nevertheless, it will be understood that various modifications may be made. For example, advantageous results may be achieved if the steps of the disclosed techniques were performed in a different sequence, or if components of the disclosed systems were combined in a different manner, or if the components were supplemented with other components. Accordingly, other implementations are contemplated within the scope of the following claims.

What is claimed is:

1. A temporary intra-cardiac pacing device comprising:
a device body (600), wherein the device body comprises a first body (135) and a second body (130) at a distal region (105), wherein the distal region comprises an electrode (605, 810) coupled to an electrode conduction wire (705) extended through a proximal end (620) of the device body, wherein the electrode is configured to contact a wall tissue and electrically connect the wall tissue to a pulse generator (610);
a coupling system comprising a loop control member (110) and extending longitudinally within the device body, and disposed between the first body and the second body; and,
a loop actuator (630) disposed at the proximal end of the device body and coupled to the loop control member, wherein:
when the loop control member is activated by tension applied from the loop actuator, the coupling system operate the first body and the second body to separate to form a loop in the distal region and to bring the electrode into contact with the wall tissue.

2. The temporary intra-cardiac pacing device of claim 1, wherein the electrode comprises a retractable fixation electrode and the electrode conduction wire comprises a fixation electrode conduction wire coupled to a slide member at the proximal end of the device body, wherein the retractable fixation electrode is disposed at a lead distal opening at the distal region, such that activation of the slide member causes the retractable fixation electrode to extend in a predetermined angle from the loop away from a distal end at the lead distal opening, such that the retractable fixation electrode is releasably coupled to the wall tissue.

3. The temporary intra-cardiac pacing device of claim 1, further comprising a plurality of electrodes.

4. The temporary intra-cardiac pacing device of claim 1, wherein the electrode comprises at least one ring electrode.

5. The temporary intra-cardiac pacing device of claim 2, wherein the retractable fixation electrode comprises a coiled end, and the slide member is configured to transmit a rotational torque to cause the coil end of the retractable fixation electrode to screw into the wall tissue.

6. The temporary intra-cardiac pacing device of claim 2, wherein the fixation electrode conduction wire is connected to two retractable fixation electrodes, wherein each of the two retractable fixation electrodes extends out of a separate lead distal openings at the distal region.

7. The temporary intra-cardiac pacing device of claim 1, wherein the loop comprises a planar gap, wherein the planar gap comprises an end-to-end separation distance between 2-2.5±1 cm.

8. The temporary intra-cardiac pacing device of claim 1, wherein the coupling system further comprises a hinge disposed near the distal region of the device body and coupled to the loop control member, wherein activation of the loop control member causes the hinge to be pulled and causes a bended expansion of the first body and the second body.

9. The temporary intra-cardiac pacing device of claim 1, wherein the coupling system further comprises a hinge thermally bonded at the distal region of the device body.

10. The temporary intra-cardiac pacing device of claim 1, wherein the coupling system further comprises a pivot adjoining a proximal end of the first body and the second body, such that activation of the loop control member causes the first body and the second body to separate at the pivot.

11. The temporary intra-cardiac pacing device of claim 1, wherein the distal region comprises a beveled portion, such that the loop is configured to form a curved shape via the loop control member.

12. An in vivo deployment platform comprising:
a device body (600), wherein the device body comprises a first body (135) and a second body (130) at a distal region, wherein the distal region comprises an end node (605, 810) coupled to a conductor (705) extended through a proximal end (620) of the device body, wherein the end node is configured to contact a wall tissue of a body cavity;
a coupling system comprising a loop control member (110) and extending longitudinally within the device body, and disposed between the first body and the second body; and,
a loop actuator (630) disposed at the proximal end of the device body and coupled to the loop control member, wherein:
when the loop control member is activated by a tension applied from the loop actuator, the coupling system force the first body and the second body to separate to form a loop at the distal region, wherein the loop is held by the applied tension, such that, when the distal region is inserted into the body cavity and the loop control member is activated, the loop at the distal region of the device body travels within the body cavity without damaging wall tissues of the body cavity.

13. The in vivo deployment platform of claim 12, wherein the end node comprises a sensor.

14. The in vivo deployment platform of claim 13, wherein the sensor comprises a camera.

15. The in vivo deployment platform of claim 12, wherein the end node comprises an actuator.

16. The in vivo deployment platform of claim 15, wherein the actuator comprises a thermal application device.

17. The in vivo deployment platform of claim 12, wherein the end node comprises a conducting ring.

18. The in vivo deployment platform of claim 12, wherein the end node comprises a retractable fixation element coupled to a fixation wire coupled to a slide member at the proximal end of the device body, wherein the retractable fixation element is disposed at a lead distal opening at the distal region, such that activation of the slide member causes the retractable fixation element to extend in a predetermined angle outwards from the loop at the lead distal opening, such that the retractable fixation element is releasably coupled to the wall tissue.

19. The in vivo deployment platform of claim 18, wherein the retractable fixation element comprises an electrode.

20. The in vivo deployment platform of claim 18, wherein the retractable fixation element comprises a coiled end, and the slide member is configured to transmit a rotational torque to cause the coil end of the retractable fixation element to screw into the wall tissue.

21. The in vivo deployment platform of claim 18, wherein the fixation wire is connected to at least a second retractable fixation element, wherein each of the retractable fixation elements extends out of separate lead distal openings at the distal region.

22. The in vivo deployment platform of claim 12, wherein the distal region comprises a beveled portion, such that the loop is configured to form a curved shape via the loop control member.

23. The in vivo deployment platform of claim 12, wherein the coupling system further comprises a hinge disposed near the distal region of the device body and coupled to the loop control member, wherein activation of the loop control member causes the hinge to be pulled and causes a bended expansion of the first body and the second body.
